(19) United States
(12) Patent Application Publication
(10) Pub. No.: US 2020/0094067 A1
(43) Pub. Date: Mar. 26, 2020

(54) Transcranial magnetic stimulation for treatment of insomnia

(71) Applicant: Khurshid Ahmad Khurshid, Newberry, FL (US)

(72) Inventor: Khurshid Ahmad Khurshid, Newberry, FL (US)

(21) Appl. No.: 16/139,058

(22) Filed: Sep. 23, 2018

(51) Int. Cl. A61N 2/00; A61B 5/00

(52) U.S. Cl. CPC A61N 2/006; A61N 2/02; A61B 5/4836

(57) ABSTRACT

Transcranial magnetic stimulation (TMS) is a non-invasive brain stimulation technique that has been approved by the Food and Drug Administration (FDA) as a treatment of depression. We tested the hypothesis that low frequency TMS exerts inhibitory effect on hyper excitable cortical state in patients with chronic insomnia and therefore could have therapeutic effect. We studied this in a pilot trial using low frequency TMS for patients with primary insomnia, We compared the change in insomnia scores between baseline and end of treatment in an open label trial with bifrontal low frequency TMS stimulation in seven patients with primary insomnia using daily stimulation of 3 weeks (15-week days). Patients between the ages of 21-65 years who meet DSM-IV criteria for Primary Insomnia were enrolled in the study. Results were positive. This study is the first of its kind to show positive effects of TMS in chronic insomnia.

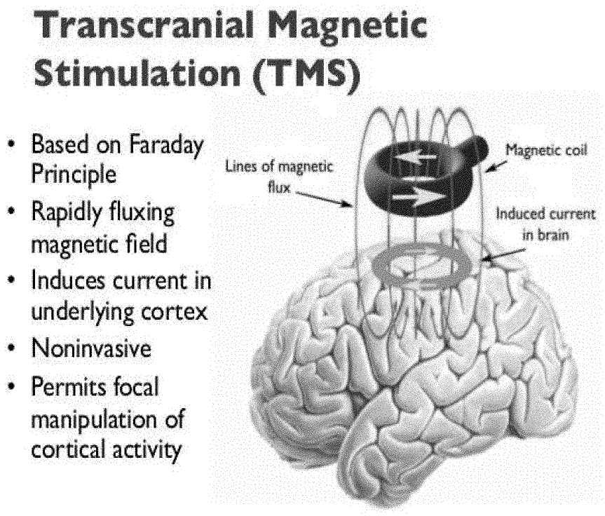

### CLASSIFICATIONS

FDA classified Transcranial magnetic stimulation under class II devices category on Aug. 25, 2011 to provide “a reasonable assurance of safety and effectiveness of these devices”.

### DESCRIPTION

Insomnia is a common clinical problem that affects more than 25 million people in the US. Insomnia exacts health and economic consequences well beyond inadequate and non-restorative sleep. It increases healthcare costs, causes or adds to medical and psychiatric comorbidities, cognitive impairments, accidents, absenteeism and reduced quality of life Patients with insomnia have impaired daytime function due to difficulty initiating sleep, difficulty maintaining sleep, or waking up early in the morning with Repetitive Transcranial magnetic stimulation (r-TMS) is a non-invasive treatment that utilizes repetitive pulses of an MRI-strength magnetic field from a coil placed over the scalp to stimulate brain tissue beneath, inducing currents that influence cortical excitability by either stimulating or inhibiting the brain activity and modulate behavior. The FDA approved r-TMS in 2008 as a treatment of patients who are treatment resistant to at least one prior antidepressant treatment. In April 2014 FDA expanded the criteria to treat depression in patients with any number of antidepressant medication courses. It has also been studied as a possible treatment for a number of other disorders, such as schizophrenia, pain, stroke, and amyotrophic lateral sclerosis (ALS). High frequency TMS (>1 Hz) has been shown to be activating whereas low frequency TMS (<1 Hz) has been shown to be inhibitory in clinical and neurophysiological studies. TMS and other neurophysiological studies have shown presence of a diffuse cortical hyper-arousal in patients with chronic insomnia^{6,7}. High frequency TMS (>1 Hz) has been shown to be activating whereas low frequency TMS (<1 Hz) has been shown to be inhibitory in clinical and neurophysiological studies.⁸ TMS has been approved as a treatment of depression in patients who have not responded to a trial of at least one antidepressant medication.

r-TMS treatment in Insomnia Disorder is appealing, as it avoids the use of polypharmacy and tolerance that develops by the use of hypnotic and sedative medications. The potential benefit of this treatment modality is that it is relatively free of, systemic side effects and well tolerated by patients. There some studies on small number of patients and healthy participants demonstrating favorable effects of r-TMS on sleep.

We used the information gained from neurophysiological studies of insomnia to the clinical treatment of insomnia using TMS as the primary treatment modality.

We compared the change in insomnia scores on Pittsburgh Sleep Quality index between baseline and end of the study with sequential bifrontal low frequency TMS in patients with primary chronic insomnia using daily stimulation over a period of 3 weeks (15-week days). Low frequency TMS (1 Hz), by exerting an inhibitory effect on cortical hyper-arousal, lead to improvement in patients with chronic insomnia as measured by PSQI. We also measured subjective and objective sleep parameters by using actigraphy (a watch like device that measured rest and activity) and compared the change in Insomnia severity index (181), a self reported insomnia measurement scale.

### REFERENCES

1. Daley M, Morin C M and LeBlanc M et al. Insomnia and its relationship to health-care utilization, work absenteeism, productivity and accidents. Sleep Medicine 2009; 10:427-438
2. Morin C M and Bence R. Chronic Insomnia. Lancet 2012; 379:1129-41
3. Daley M, Morin C M and LeBlanc M et al. The economic burden of Insomnia: direct and indirect costs for individuals with insomnia syndrome, insomnia symptoms and good sleepers. Sleep 2009; 32:55-64
4. Ohayon M M. Epidemiology of Insomnia: what we know and what we still need to learn. Sleep Med rev 2002; 6:97-111
5. Bence R M. Consequences of insomnia and its therapies. J Clin Psychiatry 2001; 62(suppl 10): 33-38
6. Bonnett M H and Arand D L. Hyper arousal and Insomnia: state of the science. Sleep Med Rev 2010 February; 14(1):9-15
7. Van der Werf Y D, Aitena E and van Dijk K D et al. Is disturbed intracortical excitability a stable trait of chronic insomnia? A study using transcranial magnetic stimulation before and after multimodal sleep therapy. Biol Psychiatry 2010 Nov. 15; 68(10): 950-5 Valero-Cabre A, Pascal-Leone A and Coubard O A. Transcranial magnetic stimulation (TMS) in basic and clinical neuroscience research. Rev Neurol 2011, 167(4): 291-316
9. Dammekens E, Vanneste S and Ost J et al. Neural correlates of high frequency repetitive transcranial magnetic stimulation improvement in post-stroke non-fluent aphasia: a case study. Neurocase 2012, Sep. 11.
10. O'Reardon J P, Solvason H B et al. Efficacy and safety of Transcranial magnetic stimulation in the acute treatment of major depression: A multisite randomized controlled trial. Biol Psychiatry 2007; 62(11); 1208-1216
11. Manconi M, Ferri R and Sagrada C, et al, Measuring the error in sleep estimation in normal subjects and in patients with insomnia. J. Sleep Res. 2010(19)478-486
12. Espie C A, MacMohan K M, Kelly H L, et al. Randomized clinical effectiveness trial of nurse-administered small-group cognitive behavior therapy for persistent insomnia in general practice. Sleep 2007; 30: 574-84
13. Khurshid K A, Gandotra K, Thapa M, Shah V and Khan R L. Relationship between depression and obstructive sleep apnea in African American patients. US Psychiatric Congress, Las Vegas, Nov. 7-10, 2011
14. Khurshid K A and Janicak P. Other than depression: treating neuropsychiatric disorders with TMS. Psychiatric Annals, 2005.
15, Khurshid K A, Holbert R D; Suryadevara U et al. Differential effects of high frequency left frontal transcranial magnetic stimulation on insomnia and depressive symptoms in patients with major depressive disoder. (Unpublished data)
16. Morin C M, Belleville G, Belanger L and Ivers H. The Insomnia severity index:psychometric indicators to detect insomnia cases and evaluate treatment response. Sleep 2011 May 1; 34 (5):601-8
17. Buysse D J, Reynolds C F and Monk T H et al., The Pittsburgh Sleep Quality index (PSQI): A new instrument for psychiatric research and practice. Psychiatry Research, 1989, 28 (2):193-213
18. Guy W. ECDEU Assessment Manual for Psychopharmacology (028 Clinical Global Impression, CGI. Rockville, Md.: National Institutes of Health, 1976:218-222
19. Montgomery S A and Asberg M. A new depression scale designed to be sensistive to change. British J Psychiatry 134(4):382-89
20. Ancoli-Israel S, Cole R and Alessi C et al. The role of actigraphy in the study of Sleep and Circadian rhythms. Sleep 2003, 26(3):342-92
21. Gross C R, Kreitzer M J and Reilly-Spong M et al. Mindfullness based stress reducation vs pharmacotherapy for primary chronic insomnia: A pilot randomized clinical trial. Explore 2011; 7(2):76-87

### BACKGROUND OF THE INVENTION

### Technical Field

It has been shown that low frequency TMS is inhibitory and high frequency TMS is stimulating to applied brain areas.

Insomnia is a heightened cortical arousal state. The rationale behind this discovery is that low frequency TMS by inhibiting the heightened arousal state exerts positive therapeutic effects.

### SUMMARY OF THE INVENTION

TMS is a non-invasive brain stimulation technique that uses magnetic stimulation. Magnetic field produced by passage of electric current through an electromagnetic field crosses scalp and skull without being impeded. It induces electrical changes in the brain areas that fall within the magnetic field generated usually few centimeters below the coil.

Low frequency TMS inhibits cortical areas that it is applied to. By inhibiting the cortical areas, it decreases hyperarousal seen in patients with insomnia. There it can have a therapeutic effect.

1. Transcranial magnetic stimulation could be utilized as a treatment option for insomnia
